(19) United States
(12) Patent Application Publication
(10) Pub. No.: US 2023/0363891 A1
(43) Pub. Date: Nov. 16, 2023

(54) NASAL PROSTHESIS AND RHINOPLASTY METHOD USING THE SAME

(71) Applicant: Sung Sig YOO, Seoul (KR)

(72) Inventor: Sung Sig YOO, Seoul (KR)

(21) Appl. No.: 18/027,876

(22) PCT Filed: Oct. 14, 2021
(86) PCT No.: PCT/KR2021/014181

§ 371 Date: Mar. 22, 2023

(30) Foreign Application Priority Data
Dec. 1, 2020 (KR) 10-2020-0165710

(51) Int. Cl. A61F 2/18; A61L 27/58; A61L 27/14

(52) U.S. Cl. CPC A61F 2/186; A61L 27/58; A61L 27/14

(57) ABSTRACT

A nasal prosthesis to be inserted into a bridge of a nose of a human body includes an insertion tip having a pointed end, a conical body portion whose diameter gradually increases rearward, and a rear end portion provided at the rear of the conical body portion. The rear end portion is formed in a hemispherical or conical shape.

### TECHNICAL FIELD

The present invention relates to a nasal prosthesis and a rhinoplasty method using the same. More particularly, the present invention pertains to a nasal prosthesis and a rhinoplasty method using the same, wherein the body of a nasal prosthesis is formed in a conical shape so that the nasal prosthesis can be easily inserted through a needle hole, protrusions are formed on the outer circumferential surface of the body so that the nasal prostheses stacked on each other are stably fixed without slippage, and a positioning thread is provided at the rear end of the nasal prosthesis so that a nasal prosthesis can be additionally inserted while preventing the position change of the already inserted nasal prosthesis.

### BACKGROUND ART

Along with the development of modern medicine, plastic surgery for giving a patient confidence and satisfaction by shaping a part of the body beautifully is increasing.

In particular, since the nose part is located at the center of the face and affects the first impression, a rhinoplasty method for making a beautiful nose is widely performed.

In most cases, a rhinoplasty method is performed to increase the height of the bridge of the nose. There are also procedures to correct a crooked nose or reduce the size of the nostrils.

As such a rhinoplasty method, there is a case in which a silicone nasal prosthesis is inserted into the bridge of the nose.

As shown in FIG. 1(a), in order to perform a rhinoplasty method using a silicone nasal prosthesis 20, an incision 12 is formed on one or both sides of the portion above the nostril 11. Alternatively, as shown in FIG. 1(b), an incision 12 is formed across both nostrils 11 and the pillar of the nose.

Subsequently, as shown in FIG. 2, a silicone nasal prosthesis 20 manufactured to fit the shape of the patient's nose is inserted through the incision 12.

After the operation is completed by inserting the silicone nasal prosthesis 20, the incision 12 is sewn with a suture.

The operation method using the silicone nasal prosthesis 20 described above has advantages in that it is inexpensive, easy to process the nasal prosthesis into a desired shape, and high in support strength of the nasal prosthesis 20, which makes it possible to reliably raise the bridge of the nose.

However, since the surface of the silicone nasal prosthesis 20 is smooth, the adhesion of the nasal prosthesis 20 to human tissue is low, which makes it difficult to fix the nasal prosthesis 20.

In addition, since the silicone nasal prosthesis 20 is an integral structure manufactured to fit the shape of the nose, it is very difficult to insert the nasal prosthesis 20 into an accurate position at once.

Accordingly, when the silicone nasal prosthesis is crookedly inserted, reoperation is required.

In addition, the silicone nasal prosthesis 20 has a problem in that the incision 12 must be sewn with a suture after being inserted into the nose, and a foreign body reaction and an inflammatory reaction may occur after insertion.

Meanwhile, as shown in FIG. 3, a method of inserting a insertion thread 30 for living body instead of the silicone implant 20 has been proposed.

The method of using the insertion thread 30 uses a thread used in a lifting procedure that pulls the skin. The insertion thread 30 is composed of a body portion 31 having a certain diameter and a plurality of spinous protrusions 32 formed on the body portion 31.

The spinous protrusions 32 are configured to be inclined in opposite directions to each other and serve to pull the skin when inserted into the skin.

According to the insertion thread 30 described above, there are advantages in that it can be inserted without forming an incision, and the support strength with respect to human tissue can be increased by the plurality of spinous protrusions 32.

In addition, by continuously inserting the insertion thread 30, a desired shape or pattern can be realized.

However, the insertion thread 30 described above has a disadvantage in that the spinous protrusions 32 are formed in opposite directions to obtain a skin lifting effect, and thus are not suitable for a rhinoplasty method.

That is, due to the spinous protrusions 32 formed in opposite directions, there is a problem in that it is not easy to push the insertion thread 30 into the nose.

Moreover, due to the spinous protrusions 32, it is difficult to stack multiple threads. In case of multiple stacking, the spinous protrusions 32 may be exposed to the outside of the nose.

As a result, experts are also experiencing difficulties in the operation, and the satisfaction of patients is not high.

In some cases, autologous cartilage and fat, which are parts of the body, are inserted into the nose.

This method has the advantage of excellent binding force and fixation force with respect to human tissue, but has the disadvantage that a separate operation must be performed to collect a part of the body.

### SUMMARY

In view of the problems inherent in the related art, it is an object of the present invention to enable a plurality of nasal prostheses to be easily inserted and stacked even through a needle hole.

Another object of the present invention is to eliminate the need to form an incision for inserting a nasal prosthesis into the bridge of the nose and to eliminate the need to sew the incision after an operation.

A further object of the present invention is to makes it possible to fix the position of a nasal prosthesis inserted into the nose or to stack additional nasal prostheses while adjusting the position thereof.

A still further object of the present invention is to increase the frictional force between a plurality of stacked nasal prostheses so that the plurality of nasal prostheses can be stably fixed without movement.

A yet still further object of the present invention is to make it possible to easily take out a nasal prosthesis inserted into the nose and to easily perform an additional operation.

According to one aspect of the present invention, there is provided a nasal prosthesis to be inserted into a bridge of a nose of a human body, including: an insertion tip having a pointed end; a conical body portion whose diameter gradually increases rearward; and a rear end portion provided at the rear of the conical body portion, wherein the rear end portion is formed in a hemispherical or conical shape.

A position adjustment thread may be further provided at the rear end portion of the prosthesis.

Net protrusions, circular protrusions, longitudinally extending protrusions, spiral protrusions, or longitudinally spaced-apart protrusions may be formed on the circumferential surface of the body portion.

The prosthesis and the position adjustment thread may be made of a non-absorbent material including silicone and nylon.

The prosthesis and the position adjustment thread may be made of an absorbent material selected from the group consisting of polydioxanone, poly-L-lactic acid, polycaprolactone, and calcium hydroxylapatite.

According to another aspect of the present invention, there is provided a rhinoplasty method for inserting a nasal prosthesis into a bridge of a nose of a human body, including: (a) forming an insertion hole by an injection needle on a front surface of a bridge of a nose (S10); (b) inserting the nasal prosthesis having a position adjustment thread into the insertion hole (S20); (c) inserting and stacking an additional prosthesis while holding the position adjustment thread of the inserted prosthesis by hand (S30); (d) cutting the position adjustment thread exposed to the outside (S40); and (e) attaching a regeneration tape to cover the insertion hole (S50).

In (b), both the body portion and the position adjustment thread of the prosthesis are positioned inside an insertion device and then inserted into the nose.

In (b), only the position adjustment thread of the prosthesis is positioned inside the insertion device and then inserted into the nose.

According to the present invention, the following effects can be obtained.

First, since the nasal prosthesis is shaped like a gentle cone with a sharp tip, multiple nasal prostheses can be inserted through the hole of a needle. As a result, unlike a silicone nasal prosthesis, there is no need to form a large incision, and there is no need to sew the incision after an operation.

Second, since the nasal prosthesis itself is provided with a positioning thread, it is possible to insert additional prostheses while holding the thread of the already inserted prosthesis with one's hand. Accordingly, it is possible to prevent the position of an already inserted nasal prosthesis from being changed due to additional insertion of a nasal prosthesis, and to adjust the position of the inserted nasal prosthesis if necessary.

Third, since the rear end of the nasal prosthesis is formed in a hemispherical or conical shape, an additional nasal prosthesis can be easily inserted through the space at the rear end of the adjacent nasal prosthesis.

Fourth, since the protrusions having a certain height are formed on the body portion of the nasal prosthesis, it is possible to stably fix the inserted nasal prosthesis by increasing the frictional force between the stacked nasal prostheses.

Fifth, after inserting the nasal prosthesis, it is possible to easily correct the height or direction of the nasal prosthesis and easily remove the inserted nasal prosthesis using the thread.

Sixth, the operation can be completed by merely attaching the skin regeneration tape to the needle mark after the insertion of the nasal prosthesis.

Seventh, it is possible to shorten the recovery time by minimizing bruising or swelling that may occur after the operation, and to prevent skin contracture from occurring.

### BRIEF DESCRIPTION OF THE DRAWINGS

FIGS. 1(a) and 1(b) are views showing incisions at the time of a conventional rhinoplasty method.

FIG. 2 is a view showing a process of inserting a silicone nasal prosthesis during a conventional rhinoplasty method.

FIG. 3 is a view showing a process of inserting an insertion thread during a conventional rhinoplasty method.

FIG. 4 is a view showing a rhinoplasty method according to the present invention.

FIGS. 5(a) to 5(e) are views showing various examples of a nasal prosthesis according to the present invention.

FIGS. 6(a) to 6(c) are views showing a rhinoplasty method according to the present invention.

FIG. 7 is a view showing a process of inserting an additional nasal prosthesis during a rhinoplasty method according to the present invention.

FIG. 8 is a view showing a process of a rhinoplasty method according to the present invention.

FIGS. 9(a) and 9(b) are views showing a state of use of an insertion device during a rhinoplasty method according to the present invention.

### DETAILED DESCRIPTION

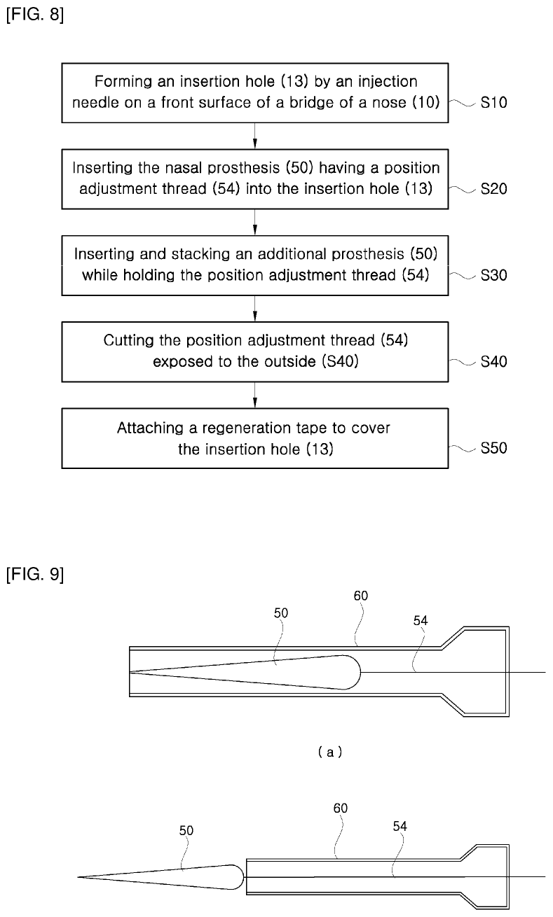

Hereinafter, a nasal prosthesis and a rhinoplasty method using the same according to the present invention will be described in detail with reference to FIGS. 4 to 9.

First, a nasal prosthesis (hereinafter simply referred to as ‘prosthesis’) according to the present invention will be described.

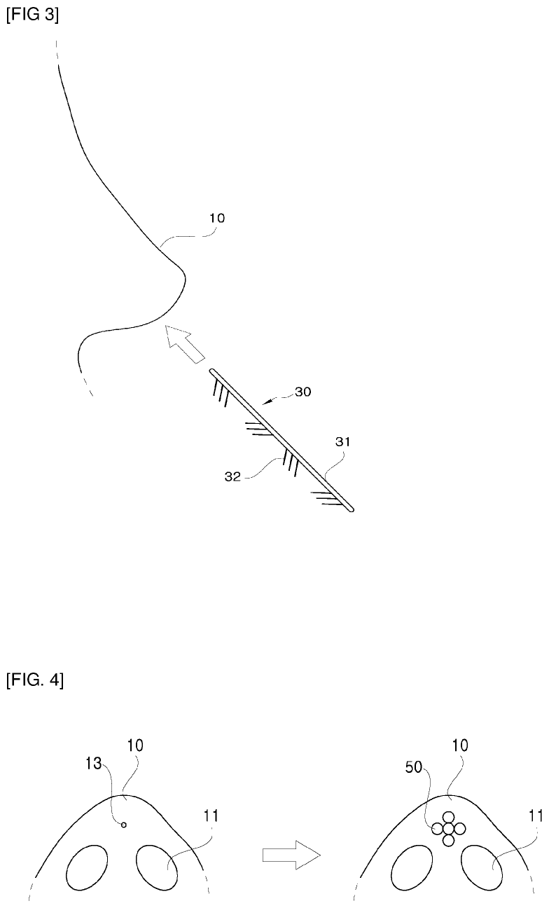

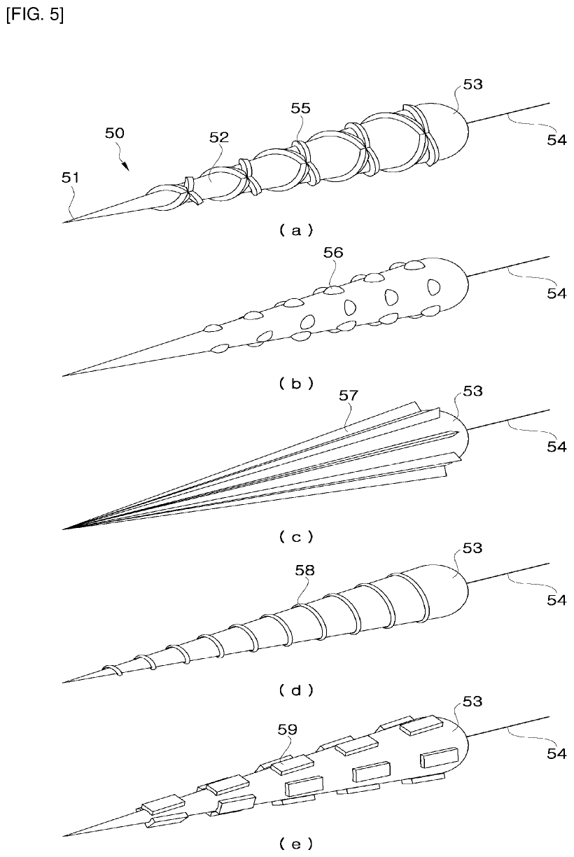

As shown in FIGS. 5(a) to 5(e), the prosthesis 50 of the present invention includes an insertion tip 51 having a pointed end, a conical body portion 52 whose diameter gradually increases rearward, and a rear end portion 53 provided at the rear of the conical body portion 52.

The reason why the body portion 52 is formed in a gentle conical shape is to consider the structure of the nose, which becomes higher as it goes down.

If the prostheses are stacked while keeping the diameter of the body portion 52 constant, the upper part of the nose becomes too high and the aesthetic appearance thereof is not good.

By using the structure of the prosthesis 50 described above, as shown in FIG. 4, a fine insertion hole 13 can be formed with an injection needle on the front surface of the bridge of the nose 10, and a plurality of prostheses 50 can be successively inserted through the insertion hole 13.

Accordingly, there is no need to form a long incision as in the case of inserting a silicone prosthesis, and there is no need to sew the incision after the operation.

In addition, the rear end portion 53 of the prosthesis 50 is preferably formed in a hemispherical or conical shape.

With the above structure, as shown in FIG. 7, a space is created between the prostheses 50 adjacent to each other, and the prosthesis 50 with a pointed end can be easily inserted through this space.

According to the present invention, a position adjustment thread 54 is further provided at the rear end portion 53 of the conical prosthesis 50.

Since the position adjustment thread 54 is not an insertion thread (see FIG. 3) used during a lifting operation, but is a thread for adjusting the position of the prosthesis, the spinous protrusions are not formed on the position adjustment thread 54.

In the prosthesis 50 according to the present invention, it is preferable that as shown in FIG. 5, protrusions having various shapes are formed on the circumferential surface of the body portion 52.

As shown in FIGS. 5(a) to 5(e), the protrusions may be formed as net protrusions 55, circular protrusions 56, longitudinally extending protrusions 57, spiral protrusions 58, and longitudinally spaced-apart protrusions 59. However, the shape of the protrusions is not limited thereto.

When the prostheses 50 are stacked, the frictional force is increased by the protrusions, so that the adjacently stacked prostheses can be stably fixed.

In addition, if the height of the protrusions is too high, it may be difficult to insert the prosthesis. Therefore, the protrusions are formed to have an appropriate size and height in consideration of the size of the prosthesis.

In addition, the prosthesis 50 and the position adjustment thread 54 of the present invention may be made of a non-absorbent material such as silicon or nylon.

Alternatively, the prosthesis 50 may be made of an absorbent material that is melted over time and absorbed into the human body.

Examples of the absorbent material preferably include polydioxanone (PDO), poly-L-lactic acid (PLLA), polycaprolactone (PCL), and calcium hydroxylapatite (CaHA).

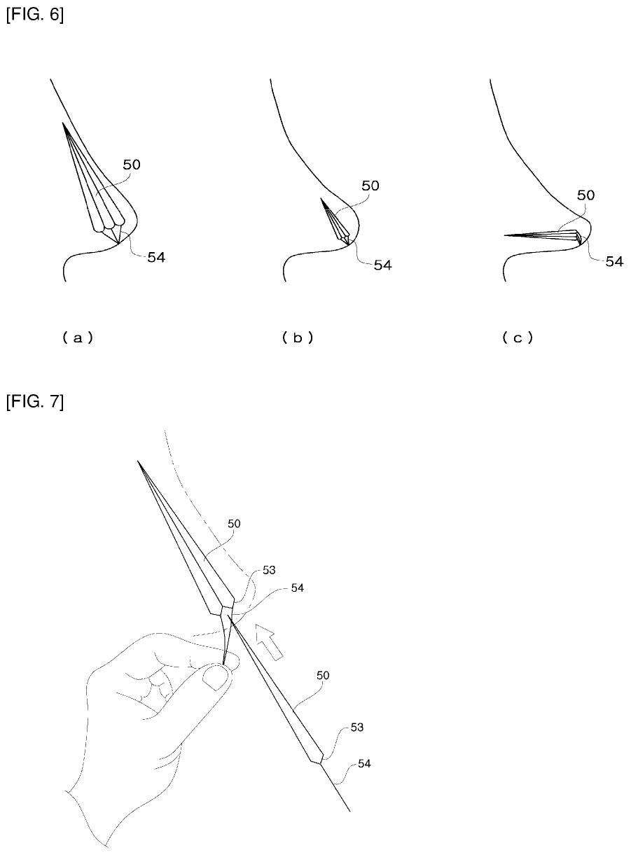

Next, a rhinoplasty method using a nasal prosthesis according to the present invention will be described with reference to FIGS. 6 to 9.

The rhinoplasty method according to the present invention is a rhinoplasty method for inserting a nasal prosthesis 50 into the bridge of the nose of a human body, including: (a) forming an insertion hole 13 by an injection needle on a front surface of a bridge of a nose 10 (S10); (b) inserting the nasal prosthesis 50 having a position adjustment thread 54 into the insertion hole 13 (S20); (c) inserting and stacking an additional prosthesis 50 while holding the position adjustment thread 54 of the inserted prosthesis 50 by hand (S30); (d) cutting the position adjustment thread 54 exposed to the outside (S40); and (e) attaching a regeneration tape to cover the insertion hole 13 (S50).

In step S10, as shown in FIG. 4, a fine insertion hole 54 is formed by an injection needle on the front surface of the bridge of the nose 10. In step S20, the prosthesis 50 provided with the position adjustment thread 54 is inserted into the insertion hole 13.

In the present invention, since the insertion tip 51 of the prosthesis 50 has a pointed end, a plurality of prostheses can be inserted through the insertion hole 13 formed by an injection needle.

Accordingly, unlike a silicone prosthesis, there is no need to form a long incision, and there is no need to sew the incision after an operation.

In step S20, the conical prosthesis 50 may be directly inserted by hand or may be inserted using an insertion device as shown in FIG. 9(a).

At this time, as shown in FIG. 9(a), both the body portion 52 and the position adjustment thread 54 of the prosthesis 50 may be positioned inside an insertion device 60 (usually called a cannula) and then inserted into the nose, or as shown in FIG. 9(b), only the position adjustment thread 54 of the prosthesis 50 may be positioned inside the insertion device 60 and then inserted into the nose.

In the case of FIG. 9(a), the insertion device 60 is inserted into the nose, the prosthesis 50 is pushed inward by a push pin (not shown), and then the insertion device 60 is pulled out while pressing the body portion of the prosthesis.

In the case of FIG. 9(b), only the position adjustment thread 54 is positioned inside the insertion device 60 and the prosthesis 50 is pushed inward by the push pin.

In step S30, as shown in FIG. 7, the additional prosthesis 50 is pushed inward through the insertion hole 13 while holding the position adjustment thread 54 of the already inserted prosthesis 50 by hand.

At this time, since the position adjustment thread 54 is held by hand, a situation in which the previously inserted prosthesis 50 is pushed inward together with the additionally inserted prosthesis 50, or the position of the previously inserted prosthesis 50 is changed does not occur.

In addition, since the rear end portion 53 of the prosthesis 50 is formed in a hemispherical or conical shape, a certain space is formed at the rear end of each of the prostheses 50 stacked adjacent to each other.

Accordingly, the prostheses 50 can be successively inserted by pushing the sharp insertion tip 51 into the space.

FIGS. 6(a) to 6(c) show examples of the rhinoplasty method according to the present invention. FIG. 6(a) shows a case where the prosthesis is inserted in the entire nose bridge, FIG. 6(b) shows a case where the prosthesis is inserted in the tip of the nose, and FIG. 6(c) shows a case where the prosthesis is inserted into the nasal column.

After inserting all of the appropriate number of prostheses 50, the threads exposed to the outside of the nose are cut away.

Subsequently, a skin regeneration tape (not shown) is attached to cover the insertion hole 13, whereby the operation is completed.

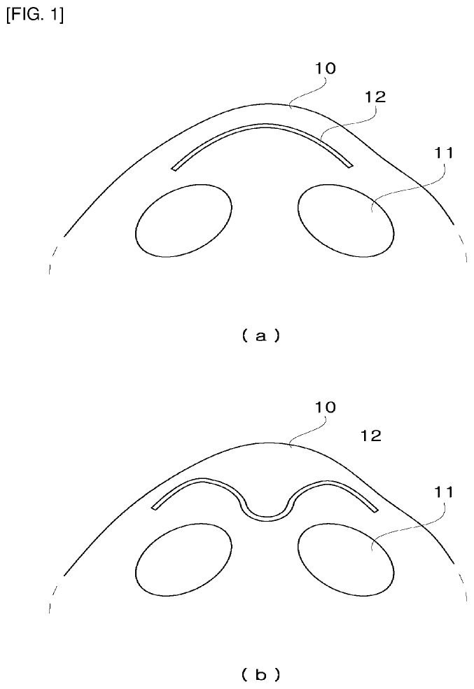

The conventional rhinoplasty method using a silicone prosthesis 20 (see FIG. 1) is inexpensive and can reliably raise the bridge of the nose. However, the incision must be made large and must be re-sewn after the operation. It is difficult to insert the prosthesis into the correct position at once. The adhesion of the prosthesis with human tissue is poor.

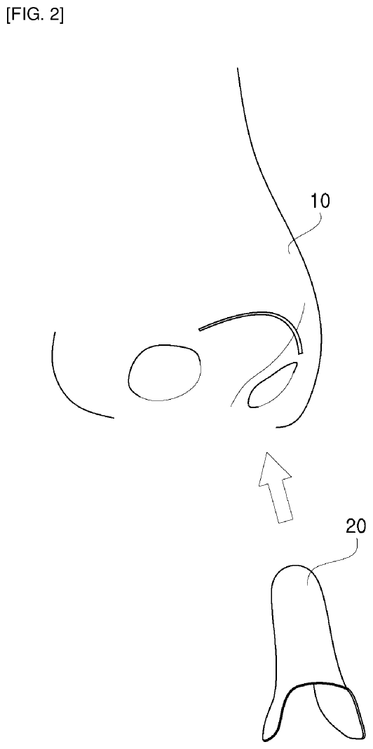

In addition, the conventional rhinoplasty method using an absorbent thread (see FIG. 2) does not have to form an incision. However, it is difficult to push the thread 30 into the nose due to the spinous protrusions 32 formed in opposite directions, and it is difficult to stack a plurality of prostheses.

The present invention utilizes only the advantages of the silicone prosthesis insertion method and the absorbable thread insertion method. The rhinoplasty method according to the present invention is a method in which the body portion of the prosthesis is formed in a conical shape at a small diameter and a plurality of prostheses are stacked.

Accordingly, as shown in FIG. 4, multiple prostheses can be inserted through the insertion hole 13 without forming an incision.

In addition, since the plurality of protrusions are formed on the outer circumferential surface of the body portion of the prosthesis, the prostheses stacked on each other can be stably fixed without slippage.

In addition, as shown in FIG. 7, since the rear end portion of the prosthesis is formed in a hemispherical or conical shape, additional prostheses with sharp ends can be successively inserted through the space between adjacent prostheses.

In addition, since the additional prosthesis is inserted while holding the thread of the already inserted prosthesis by hand, the position of the already inserted prosthesis can be prevented from being changed, and the position of the already inserted prosthesis can be adjusted if necessary.

While the preferred embodiments of the present invention have been described above, the present invention is not limited to the above-described embodiments. Various modifications and changes may be made without departing from the scope and spirit of the present invention defined in the claims.

1. A nasal prosthesis to be inserted into a bridge of a nose of a human body, comprising:
an insertion tip having a pointed end;
a conical body portion whose diameter gradually increases rearward; and
a rear end portion provided at the rear of the conical body portion,
wherein the rear end portion is formed in a hemispherical or conical shape.

2. The nasal prosthesis of claim 1, wherein a position adjustment thread is further provided at the rear end portion of the prosthesis.

3. The nasal prosthesis of claim 1, wherein net protrusions are formed on the circumferential surface of the body portion.

4. The nasal prosthesis of claim 1, wherein circular protrusions are formed on the circumferential surface of the body portion.

5. The nasal prosthesis of claim 1, wherein longitudinally extending protrusions are formed on the circumferential surface of the body portion.

6. The nasal prosthesis of claim 1, wherein spiral protrusions are formed on the circumferential surface of the body portion.

7. The nasal prosthesis of claim 1, wherein longitudinally spaced-apart protrusions are formed on the circumferential surface of the body portion.

8. The nasal prosthesis of claim 2, wherein the prosthesis and the position adjustment thread are made of a non-absorbent material including silicone and nylon.

9. The nasal prosthesis of claim 2, wherein the prosthesis and the position adjustment thread are made of an absorbent material selected from the group consisting of polydioxanone, poly-L-lactic acid, polycaprolactone, and calcium hydroxylapatite.

10. A rhinoplasty method for inserting a nasal prosthesis into a bridge of a nose of a human body, comprising:
(a) forming an insertion hole by an injection needle on a front surface of a bridge of a nose (S10);
(b) inserting the nasal prosthesis having a position adjustment thread into the insertion hole (S20);
(c) inserting and stacking an additional prosthesis while holding the position adjustment thread of the inserted prosthesis by hand;
(d) cutting the position adjustment thread exposed to the outside; and
(e) attaching a regeneration tape to cover the insertion hole.

11. The rhinoplasty method of claim 10, wherein in (b), both the body portion and the position adjustment thread of the prosthesis are positioned inside an insertion device and then inserted into the nose.

12. The rhinoplasty method of claim 10, wherein in (b), only the position adjustment thread of the prosthesis is positioned inside the insertion device and then inserted into the nose.
